(19) United States
(12) Patent Application Publication
(10) Pub. No.: US 2008/0213235 A1
(43) Pub. Date: Sep. 4, 2008

(54) Adipose Tissue Stem Cells, Perivascular Cells and Pericytes

(75) Inventors: Adam J. Katz, Charlottesville, VA (US); Shayn Peirce Cottler, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 11/995,408

(22) PCT Filed: Jul. 28, 2006
(86) PCT No.: PCT/US06/29686

§ 371 Date: Jan. 11, 2008

Related U.S. Application Data
(60) Provisional application No. 60/703,591, filed on Jul. 29, 2005.

(51) Int. Cl. A61K 35/12; C12N 5/06; A61P 9/00

(52) U.S. Cl. 424/93.21; 435/366; 435/378; 424/93.7; 435/373

(57) ABSTRACT

The present invention provides methods for growing and inducing perivascular cell differentiation of adipose tissue-derived stromal cells. The invention further provides methods for administering such adipose tissue-derived cells to a subject. The cells of the invention are useful for treating diseases, disorders, conditions, and injuries requiring new or enhanced angiogenesis, vascular remodeling, drug delivery, and tissue engineering.

### CROSS REFERENCE TO RELATED APPLICATIONS

This application claims priority under 35 U.S.C. §119(e) to U.S. Provisional Application Ser. No. 60/703,591 filed Jul. 29, 2005, the disclosure of which is incorporated by reference in its entirety herein.

### STATEMENT REGARDING FEDERALLY SPONSORED RESEARCH OR DEVELOPMENT

This invention was made with United States Government support under National Institutes of Health Grant Nos. HL-52309, HL-65958, and HL-721415. The United States Government may therefore have certain rights in the invention.

### FIELD OF THE INVENTION

The present invention relates generally to adipose tissue-derived stromal cells, and to perivascular cells and pericytes derived from adipose tissue, as well as their use.

### BACKGROUND

In recent years, the identification of mesenchymal stem cells, chiefly obtained from bone marrow, has led to advances in tissue regrowth and differentiation. Such cells are pluripotent cells found in bone marrow and periosteum, and they are capable of differentiating into various mesenchymal or connective tissues. For example, such bone-marrow derived stem cells can be induced to develop into myocytes upon exposure to agents such as 5-azacytidine (Wakitani et al., Muscle Nerve, 18 (12), 1417-26 (1995)). It has been suggested that such cells are useful for repair of tissues such as cartilage, fat, and bone (see, e.g., U.S. Pat. Nos. 5,908,784, 5,906,934, 5,827,740, 5,827,735), and that they also have applications through genetic modification (see, e.g., U.S. Pat. No. 5,591,625). While the identification of such cells has led to advances in tissue regrowth and differentiation, the use of such cells is hampered by several technical hurdles.

One drawback to the use of such cells is that they are very rare (representing as few as 1/2,000,000 cells), making any process for obtaining and isolating them difficult and costly. Of course, bone marrow harvest is universally painful to the donor. Moreover, such cells are difficult to culture without inducing differentiation, unless specifically screened sera lots are used, adding further cost and labor to the use of such stem cells. U.S. Pat. No. 6,200,606 by Peterson et al., describes the isolation of CD34+ bone or cartilage precursor cells (of mesodermal origin) from tissues, including adipose.

The presence of adult multipotent “stem” cells has been demonstrated in a large number of tissues, for example the bone marrow, blood, liver, muscle, the nervous system, and in adipose tissue. Adult “stem” cells, which in theory are capable of infinite self-renewal, have great cell plasticity, i.e. the ability to differentiate into tissues other than those for which it was believed they were destined. The properties of said cells, which are similar to those of embryonic stem cells (ES), open up considerable therapeutic perspectives especially as their use does not pose the problems of compatibility and ethics, encountered with ES cells.

Adipose tissue plays an important and overlooked role in the normal development and physiology of humans and other mammalian species. Many different kinds of fat exist. The most common type is white adipose tissue, located under the skin (subcutaneous fat), within the abdominal cavity (visceral fat) and around the reproductive organs (gonadal fat). Less common in the adult human is brown adipose tissue, which plays an important role in generating heat during the neonatal period; this type of fat is located between the shoulder blades (interscapular), around the major vessels and heart (periaortic and pericardial), and above the kidney (suprarenal).

As women mature, they develop increased amounts of mammary adipose tissue. The mammary fat pad serves as an energy source during periods of lactation. Indeed, reproductive capacity and maturation are closely linked to the adipose tissue stores of the individual. Puberty in women and men correlates closely with the production and release of leptin, an adipose tissue derived hormone, and to body fat composition. Other adipose tissue sites play a structural role in the body. For example, the mechanical fat pads in the soles of the feet provide a cushion against the impact of walking. Loss of this fat depot leads to progressive musculoskeletal damage and impaired mobility. Bone marrow fat cells are present in bone marrow to provide energy to developing blood cells within the marrow.

Bone marrow adipocytes are different than adipocytes present in adipose tissue, differing in morphology, physiology, biochemistry as well as their response to various stimulators such as insulin. Adipocytes present in bone marrow stroma may function to: 1) regulate the volume of hemodynamically active marrow; 2) serve as a reservoir for lipids needed in marrow cell proliferation, and 3) may be developmentally related to other cell lineages such as osteoblasts. White adipose tissue (i.e. body fat) in contrast, is involved in lipid metabolism and energy homeostasis (Gimble, “The Function of Adipocytes in the Bone Marrow Stroma”′ The New Biologist 2(4), 1990, pp. 304-312).

Recently, much attention has been devoted to the plasticity of different adult cell types, termed “adult stem cells” or “progenitor cells,” in injury responses and as therapeutic targets. Circulating (Iba et al., Circulation, 106:2019, 2002; Peichev et al., Hemostas. Thrombosis, and Vasc. Biol. 95:952, 2000), bone marrow derived (Asahara et al., Circ. Res. 85:221, 1999; Carmeliet and Luttun, Thrombos. & Haemostas. 86:289, 2001), and tissue derived (Majka et al., J. Clin. Invest. 111:71, 2003) progenitor cells have been implicated in the process of microvascular remodeling. A growing body of literature suggests that cells within the stromal-vascular fraction (SVF) of human adipose tissue possess previously unrecognized developmental plasticity, in vitro(Gronthos et al., J. Cell Physiol. 189:54, 2001) and in vivo (Planat-Benard et al., Circulation 109:r23, 2004; Rehman et al., Circulation 109:r52, 2004). These stromal cells have alternatively been referred to as processed lipoaspirate cells (PLA), adipose-derived stem cells, adipose-derived stromal cells, and adipose-derived mesenchymal progenitor cells. Herein, these cells are referred to as adherent adipose-derived stromal cells (ASCs) to distinguish them from SVF cells, which have not been separated based on adherence to tissue culture plastic.

Human ASCs (hASCs) have been shown to differentiate into chondrogenic, myogenic, osteogenic, and adipogenic cells in the presence of lineage-specific induction factors (Zuk et al., Tissue Engineering 7:211, 2001). Moreover, adipose-derived stromal cells have been shown to differentiate into endothelial cells (Planat-Benard et al., Circulation 109:r23, 2004; Miranville et al., Circulation 110:349, 2004), form vascular-like sprouts in matrigel (Planat-Benard et al., Circulation 109:r23, 2004), enhance neovascularization in an ischemic hindlimb model (Planat-Benard et al., Circulation 109:r23, 2004; Rehman et al., Circulation 109:r52, 2004; Miranville et al., Circulation 110:349, 2004), and secrete angiogenic and anti-apoptotic growth factors (Miranville et al., Circulation 110:349, 2004).

Those who have studied this cell type in the vascular arena have hypothesized that the pro-angiogenic activity of human adipose-derived stromal cells is a combined result of their ability to produce angiogenic growth factors and to differentiate into endothelial cells ((Planat-Benard et al., Circulation 109:r23, 2004; Rehman et al., Circulation 109:r52, 2004; Miranville et al., Circulation 110:349, 2004). However, despite these recent efforts to elucidate a role for adipose-derived stromal cells in the promotion of neovascularization, detailed in vivo behaviors of injected adipose-derived stromal cells and their primary in vivo mechanism of promoting new microvascular growth remains unclear.

Pericytes are multifunctional, polymorphic perivascular cells that lie within the microvessel basal lamina and are located on the abluminal side of endothelial cells. Endothelial cells form the inner lining of the vessel wall, and perivascular cells—referred to as pericytes, vascular smooth muscle cells or mural cells—envelop the surface of the vascular tube.

There is a long felt need in the art for methods to identify sources of cells useful for vascular growth and for remodeling of tissues, particularly sources of perivascular cells and pericytes. The present invention satisfies these needs.

### SUMMARY OF THE INVENTION

The present invention provides methods and compositions to identify and enrich ASC subpopulations based on cell surface markers consistent with the pericyte phenotype. The present invention further provides methods to administer such cells for use in enhancing processes such as vascular regrowth and remodeling.

The present invention therefore encompasses the use of ASCs, and the cells disclosed herein which arise from ASCs, for uses such as therapeutic vascularization and enhance angiogenesis, tissue engineering of vascularized constructs, long-term drug delivery, and gene therapy.

Applicants have found that human Adipose Stem Cells (ASCs) can enhance angiogenesis and increase vascularity in in vivo models of inflammation and/or tissue injury.

While prior art and/or other researchers have demonstrated and published similar findings, this earlier work suggests/implies that the biological effect observed is due to the differentiation of ASCs into endothelial cells and/or the production and release of soluble factors.

The findings of the Applicants subtly but specifically demonstrate the role of a distinctly different mechanism and cell phenotype: the perivascular cell, or pericyte. The Applicants have demonstrated this phenotype based on positional fate, morphology, cell-surface markers and biologic effects (increased microvascular density). No evidence of differentiation into endothelial cells has been found.

The Applicants disclose that there exists a subpopulation of pericytes (or pericyte precursor cells with the capacity to differentiate into this phenotype) within cultured ASC preparations. In addition, ASC-pericyte subpopulations can enhance vascularity/vascular density in vivo. This effect appears to be somewhat specific to ASCs as recent studies have revealed that human lung fibroblasts do not produce a similar effect. Additional data illustrate a strong correlation between the number of ASCs with positional and morphological characteristics of perivascular cells, and vascular density, further suggesting a need for physical cell-cell contact for effect. While mechanisms are not clear at present, the observed findings are likely related to ASC-pericyte dependent stabilization and/or maintenance of neo-microvessels, such that ASCs are essentially preventing the regression of vessel sprouts at a site of injury/inflammation/healing. The present invention further discloses methods to identify and prospectively enrich ASC subpopulations based on cell surface markers consistent with the pericyte phenotype. The present invention additionally provides methods to differentiate/increase the number of such cells in a prospective manner. The invention further provides tissue-engineered vascular constructs and methods and compositions for using such constructs in tissue engineering of various tissues to repair and replace diseased and damage tissue.

Various aspects and embodiments of the invention are described in further detail below.

### BRIEF DESCRIPTION OF THE DRAWINGS

FIG. 1. Average expression (±standard deviation) of CD31 (white) and CD144 (black) is minimal and declines to 0% by passage 5 (A). Average expression of CD34 throughout 10 cell passages (B) using three different antibodies (8G12: white, 581: black, BI-3C5: gray, n=5).

FIG. 2. Average expression (+standard deviation) of NG2 according to flow cytometry. Mean of P1 through P4 equals 15±6% (n=3).

FIG. 3. Percentage of total hASCs (black) and hLFs (gray) that exhibit pericyte-like morphologies in 48-80-treated tissues. Error bars represent standard error; *=significantly different (p≦0.05).

FIG. 4. Mesenteric tissues injected with human cells were harvested 60 days later and immunostained to visualize microvessels. A) Some DiI(+) hASCs express SMA (yellow), some do not (red), and some exhibit pericyte-like morphologies and are aligned with capillaries that express BSI-lectin (blue). B-D) Higher magnification of SMA-expressing hASCs (yellow) exhibiting pericyte-like morphologies (arrows) along BSI-lectin-positive capillaries (blue in B, green in C-D). E) Some hLFs (red) exhibit pericyte-like morphologies (arrows) along BSI-lectin-positive capillary (blue) and SMA-positive venules (green), although total vessel density is reduced compared to hASC-injected tissue. F) No DiI(+) cells are present in control, un-injected tissues (green: BSI-lectin). Scale bar=25 mm in A, B, E, F; scale bar=20 mm in C, D.

FIG. 5. Vascular length density for vehicle control (no cells: white) vs. hASC (black) vs. hLF (gray) in 48/80-treated (A) and -untreated tissues (B). Error bars represent standard error; *=significantly different (p≦0.05).

FIG. 6. At day 60 in 48-80-stimulated tissues, A) percentage of total hASCs and hLFs in the tissue that exhibit pericyte-like morphologies (but not pericyte markers), B) percentage of total hASCs in the tissue that express pericyte markers (but not morphology), and C) percentage of total hASCs in the tissue that both exhibit pericyte-like morphologies and express pericyte markers. D) Venn diagram shows the relationship between injected cell populations. Shading corresponds to populations depicted in A-C. Error bars represent standard error; *=significantly different (p≦0.05).

FIG. 7. A) Percentage of total hASCs in the tissue that exhibit pericyte-like-morphologies, and B) percentage of total hASCs that exhibit pericyte-like morphologies and also express pericyte markers for each quartile of length density (1st=lowest length density quartile; 4th=highest length density quartile). Increases in length density (from 1st to 4th quartile) are correlated with increases in the percentage of hASCs in the tissue that exhibit pericyte-like-morphologies. Error bars represent standard error; *=significantly different (p≦0.05).

### DETAILED DESCRIPTION OF THE INVENTION

### Abbreviations and Acronyms

ANOVA—analysis of variance

ASC—adherent adipose-derived stromal cell

b.w.—body weight

DiI—1,1′-dioctadecyl-3,3,3′,3′-tetramethylindocarbocyanine perchlorate

DMEM—Dulbecco's modified Eagle's medium

ES—embryonic stem cells

FBS—fetal bovine serum

hASC—human adipose-derived stromal cell

hLF—human lung fibroblast

HUVEC—human umbilical vein endothelial cells

I.P.—intraperitoneal

MSC—mesenchymal stem cell

NG2—(also called high molecular weight melanoma antigen)

PBS—phosphate buffered saline

PDGF-β—platelet derived growth factor-β

PLA—processed lipoaspirate cells

SMA—smooth muscle α-actin

SVF—stromal-vascular fraction

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “abluminal” refers to something being directed away from the lumen of a tubular structure, i.e., a blood vessel.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

By “adipose” is meant any fat tissue. The terms “adipose” and “adipose tissue” are used interchangeably herein. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, most preferably, the adipose tissue is human. A convenient source of adipose tissue is from liposuction surgery or procedures such as reduction mammoplasty, however, the source of adipose tissue or the method of isolation of adipose tissue is not critical to the invention.

Adipose-derived stem cells or “adipose-derived stromal cells” refer to cells that originate from adipose tissue.

The term “adult” as used herein, is meant to refer to any non-embryonic or non-juvenile animal. For example the term “adult adipose tissue stem cell,” refers to an adipose stem cell, other than that obtained from an embryo or juvenile animal.

As used herein, the term “affected cell” refers to a cell of a subject afflicted with a disease or disorder, which affected cell has an altered phenotype relative to a subject not afflicted with a disease or disorder.

Cells or tissue are “affected” by a disease or disorder if the cells or tissue have an altered phenotype relative to the same cells or tissue in a subject not afflicted with a disease or disorder.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

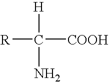

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “biocompatible,” as used herein, refers to a material that does not elicit a substantial detrimental response in the host.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder. A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

The terms “cell” and “cell line,” as used herein, may be used interchangeably. All of these terms also include their progeny, which are any and all subsequent generations. It is understood that all progeny may not be identical due to deliberate or inadvertent mutations.

The terms “cell culture” and “culture,” as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

A “compound,” as used herein, refers to a polypeptide, an isolated nucleic acid, and to any type of substance or agent that is commonly considered a chemical, drug, or a candidate for use as a drug, as well as combinations and mixtures of the above.

A “conditioned medium” is one prepared by culturing a first population of cells or tissue in a medium, and then harvesting the medium. The conditioned medium (along with anything secreted into the medium by the cells) may then be used to support the growth or differentiation of a second population of cells.

The term “delivery vehicle” refers to any kind of device or material which can be used to deliver cells in vivo or can be added to a composition comprising cells administered to an animal. This includes, but is not limited to, implantable devices, matrix materials, gels, etc.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

A “disease or disorder associated with aberrant perivascular cell activity” refers to a disease or disorder comprising either increased or decreased: perivascular cell activity; numbers of perivascular cells; or numbers of perivascular cell precursors.

As used herein, an “effective amount” means an amount of a compound or agent sufficient to produce a selected or desired effect. The term “effective amount” is used interchangeably with “effective concentration” herein.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. The terms, “feeder cells”, “feeders,” and “feeder layers” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

“Graft” refers to any free (unattached) cell, tissue, or organ for transplantation.

“Allograft” refers to a transplanted cell, tissue, or organ derived from a different animal of the same species.

“Xenograft” refers to a transplanted cell, tissue, or organ derived from an animal of a different species.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “ingredient” refers to any compound, whether of chemical or biological origin, that can be used in cell culture media to maintain or promote the growth or proliferation of cells. The terms “component,” “nutrient” and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit,” as used herein, means to suppress or block an activity or function such that it is lower relative to a control value. The inhibition can be via direct or indirect mechanisms. In one aspect, the activity is suppressed or blocked by at least 10% compared to a control value, more preferably by at least 25%, and even more preferably by at least 50%.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

The term “injury” refers to any physical damage to the body caused by violence, accident, trauma, or fracture, etc.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, the term “insult” refers to injury, disease, or contact with a substance or environmental change that results in an alteration of tissue or normal cellular metabolism in a tissue, cell, or population of cells.

The term “isolated,” when used in reference to cells, refers to a single cell of interest, or population of cells of interest, at least partially isolated from other cell types or other cellular material with which it naturally occurs in the tissue of origin (e.g., adipose tissue). A sample of stem cells is “substantially pure” when it is at least 60%, or at least 75%, or at least 90%, and, in certain cases, at least 99% free of cells other than cells of interest. Purity can be measured by any appropriate method, for example, by fluorescence-activated cell sorting (FACS), or other assays which distinguish cell types.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “modulate”, as used herein, refers to changing the level of an activity, function, or process. The term “modulate” encompasses both inhibiting and stimulating an activity, function, or process.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “pericyte-like precursor cells” as used herein refers to cells capable of differentiating into pericyte-like cells. “Pericyte-like” means that the cell expresses at least one pericyte cell marker or exhibits the morphologic characteristics of a pericyte. The term “pericyte-like” includes pericytes.

The term “perivascular-like precursor cells” as used herein refers to cells capable of differentiating into perivascular-like cells. “Perivascular-like” means that the cell expresses at least one perivascular cell marker or exhibits the morphologic characteristics of a perivascular cell. The term “perivascular-like” includes perivascular cells. Pericytes are perivascular cells.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

As used herein, the term “purified” and like terms relate to an enrichment of a cell, cell type, molecule, or compound relative to other components normally associated with the cell, cell type, molecule, or compound in a native environment. The term “purified” does not necessarily indicate that complete purity of the particular cell, cell type, molecule, or compound has been achieved during the process. A “highly purified” cell type, molecule, compound, and the like, as used herein refers to a compound that is greater than 90% pure. A “significant detectable level” is an amount of contaminate that would be visible in the presented data and would need to be addressed/explained during analysis of the forensic evidence.

A “reversibly implantable” device is one which may be inserted (e.g. surgically or by insertion into a natural orifice of the animal) into the body of an animal and thereafter removed without great harm to the health of the animal.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

Used interchangeably herein is the following pair of words—“select” and “isolate”.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In one aspect, the activity or differentiation is stimulated by at least 10% compared to a control value, more preferably by at least 25%, and even more preferably by at least 50%. The term “stimulator” as used herein, refers to any compound or agent, the application of which results in the stimulation of a process or function of interest, including, but not limited to, perivascular precursor or perivascular cell production, differentiation, and activity.

A “subject” of analysis, diagnosis, or treatment is an animal. Such animals include mammals, preferably a human.

The term “substantially pure”, as used in reference to a compound, describes a compound, e.g., a protein or polypeptide or other compound which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “tissue-engineered vascular construct” as used herein refers to formation of a tube or vessel-like structure by combining endothelial cells and adipose tissue derived stromal cells, and optionally other cells such as smooth muscles cells and fibroblasts. Such a vascular construct is to be used provide a means for transporting blood. The term “tissue-engineered construct” refers to constructing a tissue type, such as liver, muscle, etc. A “tissue-engineered construct” may further comprise vasculature or a “tissue-engineered vascular construct” to provide oxygen, nutrients, etc. to the tissue of the construct.

As used herein, the term “treating” includes prophylaxis of a specific disease, disorder, or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The terms “vascular density”, “microvascular length density”, “length density”, and “total vascular length” are used interchangeably herein and refer to the length of the vessel.

As used herein, the term “wound” relates to a physical tear or rupture to a tissue or cell layer. A wound may occur by any physical insult, including a surgical procedure.

### EMBODIMENTS

The present invention provides a perivascular cell, or pericyte, derived from adipose tissue. In one aspect, the cell is a precursor to a perivascular cell. The phenotype can be determined, inter alia, by positional fate, morphological characteristics, and other phenotypic characteristics such as cell-surface markers and biological functional characteristics such as increased microvascular density.

In one embodiment, the invention provides methods of identifying such cells and their precursors. In another embodiment, the invention provides methods of enriching populations of such cells. In one aspect, the methods of enriching such cells includes methods of inducing differentiation of the precursor cells.

In one embodiment, the present invention provides methods of increasing vascularity/vascular density in vivo using ASC derived perivascular precursor cell subpopulations. Such subpopulations may be derived from freshly derived adipose tissue or from cultured adipose tissue cells. In one aspect, the perivascular precursor cells are pericyte precursors. In one aspect, the cells of the invention are useful for microvessel maintenance. In another aspect, the cells of the invention are useful for preventing vascular regression. In yet another aspect, the cells of the invention are useful for supporting proliferation, survival, and migration, in injured vessels. Injured vessels can be caused by, inter alia, trauma and disease. In a further aspect, the cells of the invention are useful for supporting proliferation, survival, and migration of cells in new vessels.

Human adipose tissue-derived adult stromal cells represent an adult stem cell source that can be harvested routinely with minimal risk or discomfort to the patient. They can be expanded ex vivo, differentiated along unique lineage pathways, genetically engineered, and re-introduced into individuals as either autologous or allogeneic transplantation.

Methods for the isolation, expansion, and differentiation of human adipose tissue-derived cells have been reported. See for example, Burris et al 1999, Mol Endocrinol 13:410-7; Erickson et al 2002, Biochem Biophys Res Commun. Jan. 18, 2002; 290(2):763-9; Gronthos et al 2001, Journal of Cellular Physiology, 189:54-63; Halvorsen et al 2001, Metabolism 50:407-413; Halvorsen et al 2001, Tissue Eng. 7(6):729-41; Harp et al 2001, Biochem Biophys Res Commun 281:907-912; Saladin et al 1999, Cell Growth & Diff 10:43-48; Sen et al 2001, Journal of Cellular Biochemistry 81:312-319; Zhou et al 1999, Biotechnol. Techniques 13: 513-517. Adipose tissue-derived stromal cells are obtained from minced human adipose tissue by collagenase digestion and differential centrifugation [Halvorsen et al 2001, Metabolism 50:407-413; Hauner et al 1989, J Clin Invest 84:1663-1670; Rodbell et al 1966, J Biol Chem 241:130-139].

Adult human extramedullary adipose tissue-derived stromal cells represent a stromal stem cell source that can be harvested routinely with minimal risk or discomfort to the patient. Pathologic evidence suggests that adipose-derived stromal cells are capable of differentiation along multiple lineage pathways. Adipose tissue is readily accessible and abundant in many individuals. Obesity is a condition of epidemic proportions in the United States, where over 50% of adults exceed the recommended BMI based on their height.

It is well documented that adipocytes are a replenishable cell population. Even after surgical removal by liposuction or other procedures, it is common to see a recurrence of adipocytes in an individual over time. This suggests that adipose tissue contains stromal stem cells and vascular/perivascular cells and/or precursors that are capable of self-renewal.

Adipose tissue offers many practical advantages for tissue engineering applications. First, it is abundant. Second, it is accessible to harvest methods with minimal risk to the patient. Third, it is replenishable. While stromal cells represent less than 0.01% of the bone marrow's nucleated cell population, there are up to 8.6×10⁴ stromal cells per gram of adipose tissue (Sen et al., 2001, J. Cell. Biochem., 81:312-319). Ex vivo expansion over 2 to 4 weeks yields up to 500 million stromal cells from 0.5 kilograms of adipose tissue. These cells can be used immediately or cryopreserved for future autologous or allogeneic applications.

Adipose derived stromal cells also express a number of adhesion and surface proteins. These include cell surface markers such as CD9; CD29 (integrin beta 1); CD44 (hyaluronate receptor); CD49d,e (integrin alpha 4, 5); CD54 (ICAM1); CD55 (decay accelerating factor); CD105 (endoglin); CD106 (VCAM-1); CD166 (ALCAM) and HLA-ABC (Class I histocompatibility antigen); and cytokines such as interleukins 6, 7, 8, 11; macrophage-colony stimulating factor; GM-colony stimulating factor; granulocyte-colony stimulating factor; leukemia inhibitory factor; stem cell factor and bone morphogenetic protein. Many of these proteins have the potential to serve a hematopoietic supportive function and all of them are shared in common by bone marrow stromal cells.

The adipose tissue derived stromal cells useful in the methods of invention can be isolated by a variety of methods known to those skilled in the art, such as described in WO 00/5379. In a preferred method, adipose tissue is isolated from a mammalian subject, preferably a human subject. In humans, the adipose is typically isolated by liposuction. If the cells of the invention are to be transplanted into a human subject, it is preferable that the adipose tissue be isolated from that same subject to provide for an autologous transplant. Alternatively, the transplanted cells are allogeneic.

The present invention further encompasses methods for identifying perivascular-like precursor cells derived from adipose tissue. These assays and identification include, but are not limited to, use of antibodies directed against known proteins described herein or known in the art, or antibodies prepared against newly discovered proteins. The invention further encompasses identifying new antigens for such use.

Cells described herein were isolated from adipose tissue using methods previously described (Zuk et al., Tissue Engineering 7:211, 2001; Katz et al., Stem Cells 23:412, 2005). Briefly, harvested tissue was washed several times and enzymatically dissociated (Katz et al., Stem Cells 23:412, 2005; Katz et al., Adipose Tissue, In: Methods of Tissue Engineering, eds. Atala and Lanza, Academic Press, 277-286, 2002). However, one of ordinary skill in the art will appreciate that culture conditions such as cell seeding densities can be selected for each experimental condition or intended use.

Many techniques are known to those of skill in the art for measuring perivascular and pericyte cell differentiation and those not described herein are encompassed within the techniques of the invention.

Cell culture models for various disorders are useful, e.g., for testing the ability of a compound to modulate a cellular process associated with the disorder. The adipose tissue-derived stromal cells described herein are useful, e.g., for providing a pool of cells that can be differentiated at will and used in assays of such compounds.

The invention further provides for methods of using such cells in toxicological, carcinogen, and drug screening methods, as well as in therapeutic applications where microvascular growth, stability, and function is enhanced or otherwise supplanted using such cells.

In one embodiment, adipose tissue, or cells derived from adipose tissue, are subjected to varied concentrations of perivascular cell differentiation-inducing agents to induce perivascular differentiation. In one aspect, the cells are exposed to such agents in vivo. In another aspect, the agents are naturally occurring in the subject. One of ordinary skill in the art will appreciate that the amount of differentiation-inducing agent(s) used may vary according to the culture conditions, amount of additional differentiation-inducing agent used, or the number of combination of differentiation-inducing agents used when more than one agent is used to induce perivascular differentiation. The invention encompasses the differentiation of perivascular-like cells and precursors in vivo. The phrase “differentiation of perivascular-like cells and precursors” is not meant to be limited in its use and is intended to include differentiation or restriction of a multipotent cell into a cell with more limited differentiation potential as well as to differentiation in the context of maturing from an undifferentiated to a differentiated phenotype. The differentiation of such cells further relates to their function as a perivascular cell, which encompasses the proper morphology, positional fate, and function of a perivascular cells as it relates to the vessel. The invention further encompasses using perivascular cells derived from adipose tissue.

In one embodiment, perivascular cell differentiation can be negatively regulated.

Some examples of diseases, disorder, conditions, and injuries that may be treated according to the methods of the invention are discussed herein. The invention should not be construed as being limited solely to these examples, as other microvasculature-associated diseases that are at present unknown, once known, may also be treatable using the methods of the invention.

Other techniques useful for isolating and characterizing the cells described herein include fractionating cells using both perivascular cell markers and non-perivascular cell markers.

One of ordinary skill in the art will appreciate that a variety of techniques can be used to measure the differentiation and function of perivascular cells. Perivascular markers include, but are not limited to, NG2, desmin, PDGF-βreceptor, and SMA, as well as the morphological characteristics described herein and known in the art.

The development and use of tissue-engineered constructs (e.g., tissue-engineered liver, tissue-engineered bone, tissue-engineered myocardium) has been hampered, in part, by the inability to provide adequate nutrients to cells contained within the constructs where diffusion is a limiting factor (Bouhadir, 2001, J. Drug Targeting. 9:397-406; Nomi, 2002, Molecular Aspects of Medicine., 23:463-83). The goals in tissue engineering include the replacement of damaged, injured or missing body tissues with biologically compatible substitutes such as bioengineered tissues. However, due to an initial mass loss after implantation, improved vascularization of the regenerated tissue is essential. To overcome this problem, a recent thrust in the field of tissue engineering has been to identify methods for introducing a patent vasculature capable of perfusing the tissue-engineered construct with blood, thereby overcoming the limitations of oxygen and nutrient diffusion. To this end, researchers are striving to develop “vascularized tissue-engineered constructs” by incorporating endothelial cells, which can form tubes and channels within the construct that are capable of delivering blood. A main limitation in this work, however, is that it has been difficult to maintain the viability of endothelial cells and ensure their channel-forming capabilities.

It has been suggested by others that a perivascular cell coating may be necessary for endothelial cell survival and long term vessel maintenance, functionality, and patency in vascularized tissue-engineered constructs (Nomi, 2002, Molecular Aspects of Medicine., 23:463-83; Jain, 2005, Science, 307:58-621). To that end, the present invention provides hASCs as a source of perivascular cells (or perivascular-precursor cells) that can be co-cultured with endothelial cells and/or other cell types in a tissue-engineered construct to provide/encourage a patent vasculature within tissue-engineered constructs. Other cell types useful for tissue-engineered vascular constructs include, but are not limited to, smooth muscle cells and fibroblasts. Various kinds of endothelial cells are available, including, umbilical, aortic, capillary, dermal microvascular, and pulmonary artery. Not all cells are currently available from all animals of interest. Some of the endothelial cells are also available or can be obtained from different age groups, such as neonatal or adult. One of ordinary skill in the art would appreciate that a variety of growth factors and supplements can be used. For example, angiogenic growth factors including vascular endothelial cell growth factor (VEGF), basic fibroblast growth factor (bFGF), transforming growth factor-β (TGF-β), platelet derived growth factor (PDGF), the angiopoietins, and hepatocyte growth factor (HGF) can be used. In one embodiment, such a “tissue-engineered vascular construct” can be prepared first and then added to other cells or tissues to help vascularize that tissue-engineered construct. In another aspect, the perivascular cells (or perivascular-precursor cells) derived from ASCs can be combined with endothelial cells, and optionally other cells useful to form a tissue-engineered vascular construct, which are further combined with the cell types of the particular tissue type of interest for which a tissue-engineered construct is being prepared, such that the vessels form within the entire construct as that tissue construct is also forming. Additionally, such constructs could further include biocompatible matrix materials as well as three-dimensional scaffolding. One of ordinary skill in the art will recognize that the various cell types can be cultured under different conditions and combined under various parameters by varying such things as cell number, cell density, cell types, cell ratios of the different cell types used, growth media and supplements, etc.

The types of engineered tissue to which such vascular constructs of the invention can be added, include, but are not limited to, tissue-engineered liver, tissue-engineered bone, tissue-engineered myocardium, as well as any other engineered tissue in need of vasculature to perfuse the tissue with blood. Such engineered tissue constructs can be implanted directly at a site where disease or injury has effected an organ or tissue. The engineered vascular constructs of the invention can be used alone as well. Furthermore, various delivery vehicles can be used to deliver or implant such engineered vascular constructs

The present invention also encompasses pharmaceutical and therapeutic compositions comprising the adipose tissue-derived stem cells, purified perivascular cell precursors, and differentiated stromal cells and perivascular cell precursors of the present invention. In one embodiment of the invention, therapies are provided for diseases, disorders, conditions, and injuries associated with aberrant perivascular cell function, activity, numbers, or regulation. Because of the ease of isolation of ASCs and abundance of adipose tissue, these methods are superior to others using bone marrow aspirates, stem cells or circulating blood cells to produce perivascular cells or perivascular precursor cells. In one aspect, the invention provides administering to a subject in need thereof a pharmaceutical composition comprising a therapeutically effective amount of adipose tissue-derived stromal cells, perivascular-like precursors, or perivascular cells derived therefrom. Use of the terms “adipose tissue derived stromal cells”, “pericytes”, and “perivascular cells” are not intended to limit the cells to a specific step in the proliferation or differentiation process of the cell lineage. For example, a “perivascular precursor cell” can mean one that is multipotent or one that can only be induced along the pericyte cell differentiation pathway.

The present invention provides methods for administering ASCs, perivascular cells, and perivascular precursor cells to subjects in need thereof. In one aspect, the ASCs have been pretreated to differentiate into perivascular cells. In another aspect, populations of ASCs can be treated with more than one type of differentiation inducing agent or medium, or a combination of agents, which induce more than one type of differentiation. In another aspect, separate populations of ASCs, that have been pretreated with perivascular cell differentiation-inducing compounds, or no treatment at all, can be co-administered to a subject. Co-administration of different groups of cells does not necessarily mean that the ASC populations are actually administered at the same time or that the populations are combined or administered in the same composition. The invention further provides compositions and methods for administering ASCs to subjects and then inducing the ASCs to differentiate into perivascular cells by administering perivascular cell differentiation-inducing agents to the subject. In one aspect, the perivascular cell is a pericyte-like cell. In another aspect, the pericyte-like cell is a pericyte. In one aspect, the subject is a human. When more than one differentiation agent or compound is used to induce cells along the perivascular cell pathway, or when additional agents are also used to induce some of the cells to differentiate along a pathway other than perivascular cells, the various agents need not be provided at the same time. Various compounds and growth factors can be used with the cells of the invention to induce or modulate differentiation or maturation. Such differentiation inducing factors include, for example, tumor necrosis factor (TNF) and interleukin-8 (IL-8).

The invention further provides for administering to a subject the tissue-engineered vascular constructs provided herein.

In accordance with one embodiment of the invention, a method is provided for regulating tumor growth and angiogenesis, as well as diseases and disorders thereof, based on using inhibitors or stimulators of perivascular cell growth and differentiation described herein. In one aspect, angiogenesis is stimulated by administration of adipose tissue-derived perivascular cells or perivascular precursors.

The cells of the present invention may be administered to a subject alone or in admixture with a composition useful in the repair of tissue, bone, and vascular injury and defects. Such compositions include, but are not limited to bone morphogenetic proteins, hydroxyapatite/tricalcium phosphate particles (HA/TCP), gelatin, poly-L-lysine, and collagen.

Non-synthetic matrix proteins like collagen, glycosaminoglycans, and hyaluronic acid, which are enzymatically digested in the body, are useful for delivery to bone areas (see U.S. Pat. Nos. 4,394,320; 4,472,840; 5,366,509; 5,606,019; 5,645,591; and 5,683,459) and are suitable for use with the present invention. Other implantable media and devices can be used for delivery of the cells of the invention in vivo. These include, but are not limited to, sponges, such as those from Integra, fibrin gels, scaffolds formed from sintered microspheres of polylactic acid glycolic acid copolymers (PLAGA), and nanofibers formed from native collagen, as well as other proteins. The cells of the present invention can be further combined with growth factors, nutrient factors, pharmaceuticals, calcium-containing compounds, anti-inflammatory agents, antimicrobial agents, or any other substance capable of expediting or facilitating vascular growth, stability, and remodeling.

The compositions of the present invention can also be combined with inorganic fillers or particles. For example for use in implantable grafts the inorganic fillers or particles can be selected from hydroxyapatite, tri-calcium phosphate, ceramic glass, amorphous calcium phosphate, porous ceramic particles or powders, mesh titanium or titanium alloy, or particulate titanium or titanium alloy.

In one embodiment, a composition comprising the cells of the invention is administered locally by injection. Compositions comprising the cells can be further combined with known drugs, and in one embodiment, the drugs are bound to the cells. These compositions can be prepared in the form of an implantable device that can be molded to a desired shape. In one embodiment, a graft construct is prepared comprising a biocompatible matrix and one or more cells of the present invention, wherein the matrix is formed in a shape to fill a gap or space created by the removal of a tumor, injured, or diseased tissue. In one aspect, the graft construct is a tissue-engineered vascular construct.

The cells can be seeded onto the desired site within the tissue to establish a population. Cells can be transferred to sites in vivo using devices such as catheters, trocars, cannulae, stents (which can be seeded with the cells), etc.

The present invention thus provides methods and compositions for delivering incredibly large numbers of perivascular precursors or perivascular cells derived from adipose tissue stromal cells for the procedures and treatments described herein. Additionally, for diseases that require perivascular cell or perivascular precursor cell infusions, adipose tissue harvest is minimally invasive, yields many cells, and can be done repeatedly

The present invention encompasses the preparation and use of immortalized cell lines, including, but not limited to, perivascular precursor, pericyte precursor, and cell lines or adipose tissue-derived cell lines capable of differentiating into perivascular-like cells. Various techniques for preparing immortalized cell lines are known to those of ordinary skill in the art. The present invention also encompasses the preparation and use of cell lines or cultures for testing or identifying agents for their effects on bone via effects on vascular growth and differentiation. In one aspect, the invention encompasses a system to screen agents useful for treating microvascular-associated diseases, disorders, conditions and injury. The present invention further encompasses compounds, which are identified using any of the methods described herein. Such compounds may be formulated and administered to a subject for treatment of the diseases, disorders, conditions, and injuries disclosed herein.

In one embodiment, genes of interest can be introduced into cells of the invention. In one aspect, such cells can be administered to a subject. In one aspect, the subject is afflicted with a bony disease, disorder, condition, or injury. In one aspect, the cells are modified to express exogenous genes or are modified to repress the expression of endogenous genes, and the invention provides a method of genetically modifying such cells and populations. In accordance with this method, the cell is exposed to a gene transfer vector comprising a nucleic acid including a transgene, such that the nucleic acid is introduced into the cell under conditions appropriate for the transgene to be expressed within the cell. The transgene generally is an expression cassette, including a coding polynucleotide operably linked to a suitable promoter. The coding polynucleotide can encode a protein, or it can encode biologically active RNA (e.g., antisense RNA or a ribozyme). Thus, for example, the coding polynucleotide can encode a gene conferring resistance to a toxin, a hormone (such as peptide growth hormones, hormone releasing factors, sex hormones, adrenocorticotrophic hormones, cytokines (e.g., interferons, interleukins, lymphokines), a cell-surface-bound intracellular signaling moiety (e.g., cell adhesion molecules, hormone receptors), a factor promoting a given lineage of differentiation, etc.

In addition to serving as useful targets for genetic modification, many cells and populations of the present invention secrete various polypeptides. Such cells can be employed as bioreactors to provide a ready source of a given hormone, and the invention pertains to a method of obtaining polypeptides from such cells. In accordance with the method, the cells are cultured under suitable conditions for them to secrete the polypeptide into the culture medium. After a suitable period of time, and preferably periodically, the medium is harvested and processed to isolate the polypeptide from the medium. Any standard method (e.g., gel or affinity chromatography, dialysis, lyophilization, etc.) can be used to purify the hormone from the medium, many of which are known in the art.

In other embodiments, cells (and populations) of the present invention secreting polypeptides can be employed as therapeutic agents. Generally, such methods involve transferring the cells to desired tissue, either in vitro or in vivo, to animal tissue directly. The cells can be transferred to the desired tissue by any method appropriate, which generally will vary according to the tissue type.

Compositions comprising cells of the invention can be employed in any suitable manner to facilitate the growth and differentiation of the desired tissue. For example, the composition can be constructed using three-dimensional or stereotactic modeling techniques. To direct the growth and differentiation of the desired structure, the composition can be cultured ex vivo in a bioreactor or incubator, as appropriate. In other embodiments, the structure is implanted within the host animal directly at the site in which it is desired to grow the tissue or structure. In still another embodiment, the composition can be engrafted onto a host, where it will grow and mature until ready for use. Thereafter, the mature structure (or anlage) is excised from the host and implanted into the host, as appropriate.

Matrices suitable for inclusion into the composition can be derived from various sources. As discussed above, the cells, matrices, and compositions of the invention can be used in microvasculature tissue engineering and regeneration. Thus, the invention pertains to an implantable structure (i.e., an implant) incorporating any of these inventive features. The exact nature of the implant will vary according to the intended use. The implant can be, or comprise, as described, mature or immature tissue. Thus, for example, one type of implant can be a bone implant, comprising a population of the inventive cells that are undergoing (or are primed for) perivascular or pericyte differentiation, optionally seeded within a matrix material. Such an implant can be applied or engrafted to encourage the generation or regeneration of mature bone tissue within the subject.

One of ordinary skill in the art would appreciate that there are other carriers useful for delivering the cells of the invention. Such carriers include, but are not limited to, calcium phosphate, hydroxyapatite, and synthetic or natural polymers such as collagen or collagen fragments in soluble or aggregated forms. In one aspect, such carriers serve to deliver the cells to a location or to several locations. In another aspect, the carriers and cells can be delivered either through systemic administration or by implantation. Implantation can be into one site or into several sites.

In certain useful applications, compounds are screened specifically for potential toxicity. Cytotoxicity can be determined in the first instance by the effect on cell viability, survival, morphology, and leakage of enzymes into the culture medium. Other methods to evaluate toxicity include determination of the synthesis and secretion of target proteins of interest and induction of apoptosis (indicated by cell rounding, condensation of chromatin, and nuclear fragmentation). DNA synthesis can be measured using assays such as tritiated-thymidine or BrdU incorporation. Effects of a drug on DNA synthesis or structure can be determined by measuring DNA synthesis or repair. Aberrant DNA synthesis, especially at unscheduled times in the cell cycle, or above the level required for cell replication, is consistent with a drug effect. Unwanted effects can also include unusual rates of sister chromatid exchange, determined by metaphase spread (see pp 375-410 of Vickers (1997) In vitro Methods in Pharmaceutical Research Academic Press).

In one embodiment, the cells of the invention are used to screen factors that promote perivascular cell differentiation or promote proliferation and maintenance of such cells, or precursors, in long-term culture. Assays are known in the art for measuring perivascular cell differentiation and proliferation. Such factors can be known drugs, agents, proteins, nucleic acids, etc., which can be used to treat cells using the assays and methods described herein or which are known in the art.

In another embodiment, cells of the invention are used to screen factors that inhibit perivascular cell differentiation.

In yet another embodiment, differentiated or undifferentiated cells of the invention are used to screen factors that modulate perivascular production, differentiation, function, and activity.

In general, methods for the identification of a compound which effects the differentiation, production, activity, or function of a cell of the invention, include the following general steps:

The test compound is administered to a cell, tissue, sample, or subject, in which the measurements are to be taken. A control is a cell, tissue, sample, or subject in which the test compound has not been added. A higher or lower level of the indicator or parameter being tested, i.e., cell number, perivascular cell production, differentiation, activity, function, etc., in the presence of the test compound, compared with the levels of the indicator or parameter in the sample which was not treated with the test compound, is an indication that the test compound has an effect on the indicator or parameter being measured, and as such, is a candidate for modulation of the desired activity. Test compounds may be added at varying doses and frequencies to determine the effective amount of the compound which should be used and effective intervals in which it should be administered. In another aspect, a derivative or modification of the test compound may be used.

In one embodiment, antibodies can be used to identify perivascular cell or perivascular precursor cell markers. In another embodiment, antibodies directed against proteins of cells of the invention can be used to modulate the activity of the proteins. The invention encompasses preparing such antibodies.

The antibodies of the present invention can be combined with a carrier or diluent to form a composition. In one embodiment, the carrier is a pharmaceutically acceptable carrier. In another embodiment, the antibodies are linked to a solid support. In yet another embodiment, the antibodies are linked to a detectable marker.

Under suitable conditions, a colorimetric reporter molecule forms a color or changes color, a fluorescent reporter molecule fluoresces or changes fluorescence, and a chemiluminescent reporter molecule chemiluminesces, or emits light due to a chemical reaction. Horseradish peroxidase (HRP) may be considered to be a colorimetric reporter molecule. An antibody-HRP conjugate causes precipitation of a colored substrate where the antibody binds to the corresponding antigen.

A reporter molecule may be an enzyme or an enzyme substrate. If the reporter molecule is an enzyme, the corresponding enzyme substrate is added after the antibody is allowed to bind to the corresponding antigen. If the reporter molecule is an enzyme substrate, the corresponding enzyme is added. Reaction between the enzyme and the enzyme substrate gives rise to the formation of a color, a change in color, fluorescence, a change in fluorescence, or chemiluminescence.

In one embodiment, the antibodies are labeled either directly or indirectly, using an immunofluorescence compound and techniques known to those skilled in the art. In the direct method, the antibodies are labeled directly with a fluorochrome. In the indirect method, the fluorochrome is attached to a secondary antibody that recognizes the primary antibody. In one embodiment, the primary antibodies are monoclonal antibodies that have been directly conjugated to a fluorochrome.

The indirect method has the advantage that it can amplify the fluorescent signal by binding more fluorochrome at the antigen site. Therefore, its potential fluorescent signal may be stronger than the direct method, especially at low antibody-conjugate concentrations. A drawback of the indirect method is that it employs two separate steps of antibody addition.

The direct method has the advantage that it reduces the number of washing steps and is quicker. The use of a single labeled immunoreagent also reduces the background fluorescence by eliminating non-specific binding of the secondary antibody. One possible drawback of using a single labeled immunoreagent is that at low antibody-antigen ratios, the fluorescent signal may be lower than that in the indirect method.

Antibodies may be generated using methods that are well known in the art. For instance, U.S. patent application Ser. No. 07/481,491, which is incorporated by reference herein in its entirety, discloses methods of raising antibodies to specific proteins. For the production of antibodies, various host animals, including but not limited to rabbits, mice, and rats, can be immunized by injection with a specific polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For the preparation of monoclonal antibodies, any technique which provides for the production of antibody molecules by continuous cell lines in culture may be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) may be employed to produce human monoclonal antibodies. In another embodiment, monoclonal antibodies are produced in germ-free animals utilizing the technology described in international application no. PCT/US90/02545, which is incorporated by reference herein in its entirety.

In accordance with the invention, human antibodies may be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be employed; such antibodies are within the scope of the present invention. Once specific monoclonal antibodies have been developed, the preparation of mutants and variants thereof by conventional techniques is also available.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Bio1.248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18−silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for analogs of proteins. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The invention also includes a kit comprising cells, compositions, and compounds of the invention and an instructional material which describes administration to a subject.

As used herein, an “instructional material” includes a publication, a recording, a diagram, or any other medium of expression which can be used to communicate the usefulness of the cells or compounds of the invention in the kit for effecting alleviation of the various diseases, disorders, conditions, or injuries recited herein. Optionally, or alternately, the instructional material may describe one or more methods of alleviation the diseases or disorders in a tissue of a subject.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

The studies described herein investigate, at the single cell level, the potential of hASCs to contribute to microvascular growth when injected in vivo, and we have found that injected hASCs migrate to the abluminal surface of microvessels and alter their cell morphology to conform to the curvature of the microvessel in a manner that is consistent with perivascular (and not endothelial) cell behavior. Also examined herein is the expression of various perivascular cell markers, focusing on the pericyte population, a cell population defined by their morphological structure around microvessels with cell processes extending beneath the basement membrane. Since pericytes have been suggested to contribute to microvessel growth and maintenance, it was determined whether hASCs function as microvascular support cells by analyzing their perivascular investment in relation to changes in total vascular density. The present application discloses a new role for human adipose-derived stromal cells in the promotion of vascular stability and the enhancement of microvascular growth in vivo.

Methods

Isolation, Culture, and Labeling of hASCs

Subcutaneous adipose tissue was obtained from 14 female patients undergoing elective surgical procedures in the Department of Plastic Surgery, University of Virginia (age range: 30-56; mean=42 years old). The University of Virginia's Human Investigation Committee approved tissue harvest protocols. Adipose tissue came either from intraoperative suction lipectomy (n=6) or from laboratory liposuction of panniculectomy specimens (n=8).

Cells were isolated from adipose tissue using methods previously described (Zuk et al., Tissue Engineering 7:211, 2001; Katz et al., Stem Cells 23:412, 2005). Briefly, harvested tissue was washed several times and enzymatically dissociated (Katz et al., Stem Cells 23:412, 2005; Katz et al., Adipose Tissue, In: Methods of Tissue Engineering, eds. Atala and Lanza, Academic Press, 277-286, 2002). Dissociated tissue was filtered to remove debris, and the resulting cell suspension was centrifuged. Pelleted stromal cells were recovered and washed several times. Contaminating erythrocytes were lysed with an osmotic buffer, and the stromal cells were plated onto tissue culture plastic. Cultures were washed with buffer 24-48 hours after plating to remove unattached cells, and then re-fed with fresh medium. Plating and expansion medium consisted of DMEM:F12 with 10% FBS and antibiotic-antimycotic. Cultures were maintained at 37° C. with 5% CO2 and fed three times per week.

Cells were grown to confluence after the initial plating (P=0), typically within 10-14 days. Adherent cells were released with 0.5% trypsin-EDTA and re-plated at 2,000 cells/cm² (passage 1; P1). Cell cultures were passaged every 7-8 days until analysis. All cells used for injection studies were between passage 2 and 4, corresponding to approximately 11 or fewer total population doublings.

One day prior to injection, cells were labelled with the fluorescent marker DiI (5 μM) according to manufacturer's instructions (Molecular Probes, OR). Cells were rinsed, trypsinized, counted, and resuspended for injection. The use of DiI as a label for identifying progenitor cells in vivo has been well documented (Iwaguro et al., Circulation 105:732, 2002; Weber et al., European J. Cardio-Thoracic Surg. 26:137, 2004; Mothe and Tator, Neuroscience 131:177, 2005; Barresi et al., Cancer Gene Therapy 10:396, 2003). DiI-labeled hASCs were co-cultured with unlabeled hASCs and HUVECs to confirm that DiI transfer between cells did not occur. Furthermore, plated hASCs were labeled with DiI and maintained in culture to confirm that DiI fluorescence did not diminish visibly over time or with cell division.

Immuno-Phenotypic Characterization of hASCs

The vascular-related cell surface phenotype of hASCs was analyzed using flow cytometry. Freshly isolated (SVF) cells and cultured hASCs were evaluated for their expression of the following cell surface proteins over time in culture: CD31 (Pecam-1) (BD Bioscience, CA), CD34 (BD Bioscience; Santa Cruz Biotechnology, Inc., CA; and Caltech Labs, CA), CD62P(P-Selectin) (eBioscience, San Diego, Calif.), CD106 (VCAM-1) (eBioscience, CA), CD133 (Miltenyi Biotech, CA), and CD144 (VE-Cadherin) (BD Bioscience), CD140b (PDGF-β receptor) (BD Bioscience), and NG2 (Beckman Coulter, FL). Flow cytometry was performed on a Becton Dickinson FACS Calibur, as previously described (Katz et al., Stem Cells 23:412, 2005). A minimum of 10,000 events were counted for each analysis. HLA-ABC was used as a positive control for each flow trial.

Animal Studies

Experiments were performed using sterile techniques according to the guidelines of the University of Virginia Animal Care and Use Committee. Seventy-two male, nude rats (NCI) weighing 250±20 grams were divided into six study groups: 1) hASC injection (1×10⁶ cells); 2) hASC injection (1×10⁶ cells) and compound 48-80 stimulation; 3) human lung fibroblast (hLF) injection (1×10⁶ cells); 4) hLF injection (1×10⁶ cells) and 48-80 stimulation; 5) vehicle control (sterile PBS); and 6) vehicle control and 48-80 stimulation.

Stimulation of Microvascular Remodeling with Compound 48-80 and Cell Injection

Compound 48-80 (condensation product of N-methyl-p-methoxyphenylethylamine with formaldehyde; Sigma, MO) is a pharmacological agent known to act specifically on mast cells by inducing degranulation. Injection of Compound 48-80 into the rat mesentery stimulates well-characterized microvascular growth and remodeling in the mesenteric vasculature (Nehls et al., Cell & Tissue Res 270:469, 1992; Norrby et al., Virchows Archiv. B. Cell Pathology. 52:195, 1986), and this small animal model is a well established assay for studying cellular and molecular mechanisms of angiogenesis (Anderson et al., J. Histochem. & Cytochem. 52:1063, 2004; Bocci et al., Cancer Chemotherapy & Pharmacol 43:205, 1999). Compound 48-80 was injected I.P.(1 ml/100 gram animal weight) in 0.9% sterile NaCl on the first 5 consecutive study days. Two doses of each concentration (100, 200, 300, and 400 μg/ml) were administered per day separated by 8 hours on the first four days. On day 5, rats in these study groups received a single dose of 500 μg/ml. To rule out any direct effects of Compound 48-80 on hASC survival, proliferation, and differentiation, cultured hASCs (P=2) were exposed to either 100 or 5 μg/ml of Compound 48-80 in the media for six days. Cell counts and flow cytometry confirmed that Compound 48-80 (at both concentrations) did not directly promote HASC survival or NG2 expression, but in fact, reduced cell proliferation and viability (data not shown).

On day 4 of 48-80 injections, 1×10⁶ DiI labeled hASCs or hLFs (WI-38 cell line, No. CCL-75, ATCC) in 0.5 ml sterile PBS were injected I.P. using syringes with 25⅜ G, 0.5 inch needles.

Harvesting of Mesenteric Tissue

Rats were anesthetized with intramuscular injections of ketamine (80 mg/kg b.w.), atropine (0.08 mg/kg b.w.), and xylazine (8 mg/kg b.w.). Six mesenteric windows were harvested from each animal at 10, 30, or 60 days after cell injection. Tissues were whole-mounted on gelatin-coated slides.

Immunohistochemistry

Tissues were washed in PBS+0.1% saponin 3 times for 10 minutes and immunolabeled with lectin from Bandeiraea simplicifolia (BSI-lectin) FITC-conjugate (1:100, Sigma Biosciences, St. Louis, Mo.) or Alexa Fluor 647-conjugate (1:100, Molecular Probes, OR), and/or antibody to smooth muscle α-actin (SMA) using purified FITC-conjugated clone 1A4 mouse monoclonal anti-SMA (1:500, Sigma Biosciences, St. Louis, Mo.), diluted in PBS buffer containing 0.1% saponin and 2% bovine albumin (Fisher Scientific) at pH 7.4 (incubation for 1 hour at room temperature). Tissues were also stained with perivascular cell markers (Gerhardt and Betsholtz, Cell Tissue Res. 314:15, 2003), including antibodies to: 1) NG2 (1:150, rabbit polyclonal, Chemicon, Int.), Desmin (1:100, mouse anti-human clone D33, DAKO, Denmark), and PDGF-βR (1:100, rabbit polyclonal, Santa Cruz Biotechnology, CA). Cy2-conjugated secondary antibodies were applied for 1 hour at room temperature: 1 & 3) goat anti-rabbit IgG, and 2) goat anti-mouse IgG-fab fragment (1:100, Jackson Immunoresearch, PA). In the final wash cycle, Hoechst 33342 (1×10⁻⁶ mM) or TOTO-3 (1:1000; Molecular Probes, OR) was added for visualization of nuclei.

Inage Acquisition and Data Analysis

Mesenteric tissues were examined with a Nikon Eclipse TE2000-E microscope equipped with confocal accessories (Nikon D-Eclipse C1) using 20× Nikon water/oil immersion and 60× Nikon oil immersion objectives. Images were digitized and analyzed using Scion Image software version 4.0.2 (Scion Corporation, Frederick, Md.). The number of DiI-positive cells per tissue area and total microvessel length were quantified. Nuclei were visualized with TOTO-3 and Hoechst 33342 to confirm the presence of DiI-labeled hASCs and hLFs.

Statistical Analysis

Results are presented in the form of mean ±standard error. Comparisons were made using the statistical analysis tools provided by SigmaPlot 5.0 (SPSS, Inc., Chicago, Ill.) Data were tested for normality and analyzed by one-way ANOVA followed by non-paired Tukey's T-test. Significance was asserted at p<0.05.

Results

Immuno-Characterization of hASCs

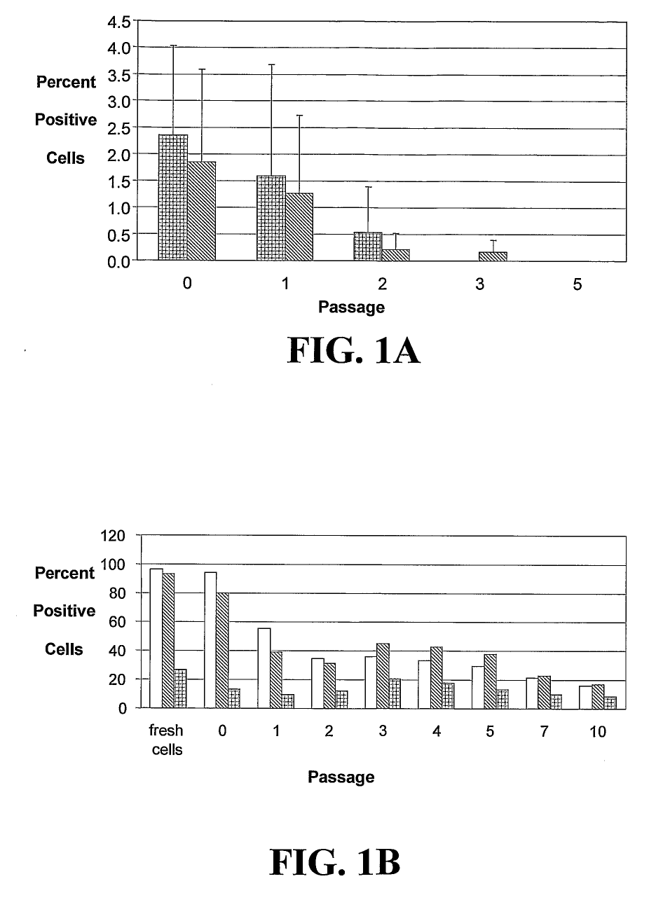

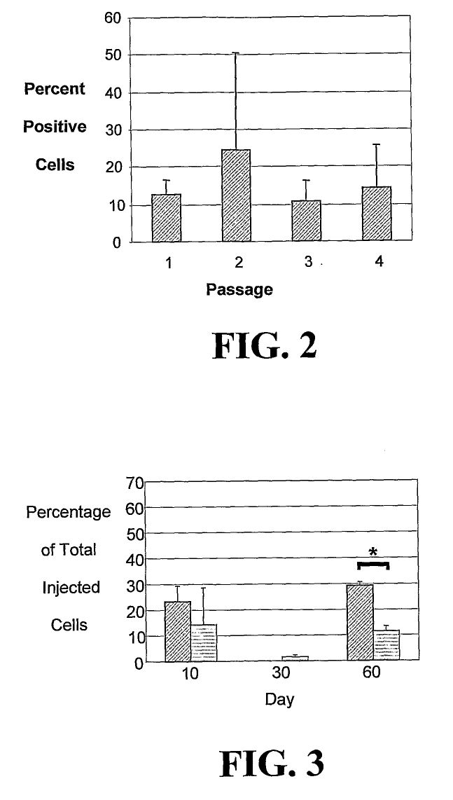

The vascular-related cell surface profile of hSVF and hASCs were analyzed using flow cytometry and or immunohistochemistry (SMA, Desmin). A very small percentage (2.5% or less) of early passage hASCs stain positively for markers of mature and/or activated endothelial cells (CD 31, CD144, CD 62P, and CD 106), and these phenotypic markers become undetectable by passage 2/3 (FIG. 1A). Under the described isolation and culture conditions, less than 1% of hASCs stain for the progenitor/stem cell marker CD133, whereas 9-97% of them stain positively for CD34, with specific results depending on both the length in culture and the specific antibody utilized FIG. 1B). Finally, hASCs consistently stain for several markers consistent with a perivascular phenotype (Gerhardt and Betsholtz, Cell Tissue Res. 314:15, 2003) including NG2 (FIG. 2), PDGF-β receptor (CD140b; 76-97% by flow; mean=90%), and smooth muscle actin (SMA; approximately 25% by immunocytochemistry—data not shown). Negligible staining was observed for desmin (data not shown).

Total Numbers of Injected hASCs and hLFs is Dependent on Stimulus and Time

The total number of hASCs in the mesenteric tissue exhibits a biphasic response, decreasing from days 10 to 30 and increasing from days 30 to 60 (Table 1). In contrast, the total number of hLFs in mesenteric tissues remains relatively constant, yet is significantly diminished when averaged over all three analysis time points compared to hASCs. When hASCs or hLFs are injected into tissues that have not received Compound 48-80, they are initially (day 10) undetectable in number (Table 1), suggesting that either Compound 48-80 or the pro-angiogenic, inflamed tissue environment created by this stimulus promotes human cell survival in vivo. The former explanation can be ruled out by vitro studies (data not shown) that indicate Compound 48-80 negatively impacts hASC survival and proliferation directly, thereby suggesting that a pro-angiogenic environment created by Compound 48-80 in the early stages enhances cell numbers in vivo. Whereas hASC numbers remain very low in tissues lacking 48-80 stimulation at days 30 and 60, hLF numbers increase with time but are not statistically different from hLF numbers in 48-80-treated tissue at any corresponding time point.

TABLE 1
Total number of injected cells in 48-80-treated
and un-treated tissues.

| Cell Type | Treatment Type | Day 10 | Day 30 | Day 60 |
|---|---|---|---|---|
| hASC | +48/80 | 202 ± 58 | 9 ± 5 | 63 ± 5 |
| | −48/80 | 0 ± 0 | 6 ± 6 | 4 ± 4 |
| hLF | +48/80 | 21 ± 9 | 17 ± 3 | 21 ± 3 |
| | −48/80 | 0 ± 0 | 5 ± 2 | 55 ± 6 |

hASCs Exhibit Perivascular Morphology In Vivo

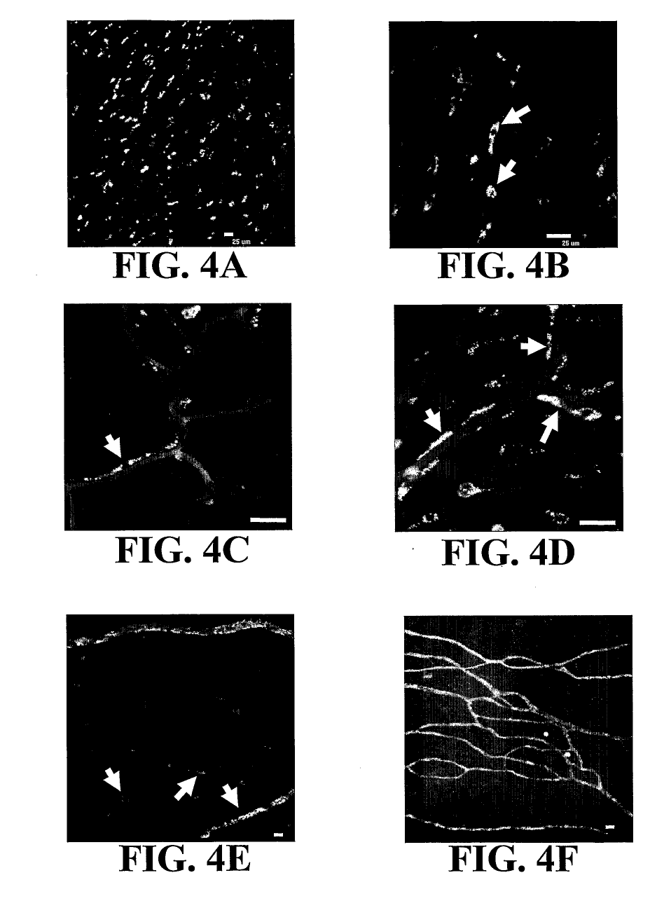

As early as 10 days following injection, hASCs are visible in mesenteric tissue and upwards of 20% exhibit pericyte-like morphology (FIG. 3). For the purposes of obtaining and analyzing data, a cell was arbitrarily said to have pericyte-like morphology if it had processes extend along vessels in a manner that conforms to the curvature of the vessel and whose cell bodies were no more than about 5 μm from the abluminal surface of the endothelium. The distance was merely set as a reference point for obtaining and analyzing data.

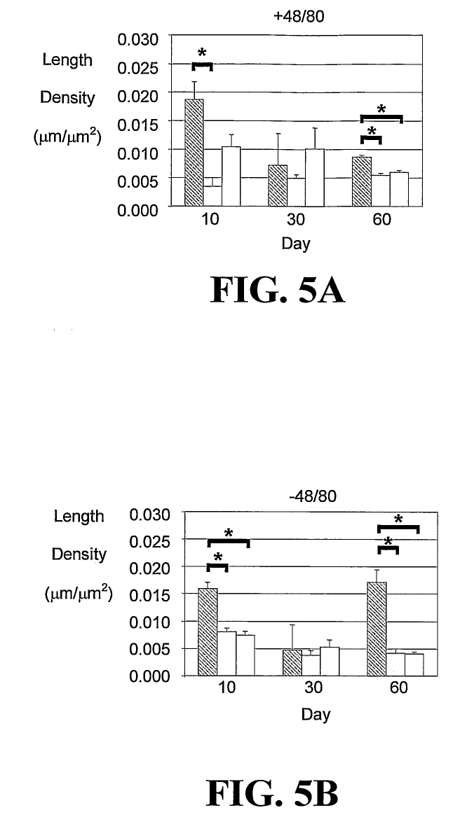

The cell behavior quantified herein is distinct from smooth muscle cell morphology, which is characterized by wrapping of the smooth muscle cell around the abluminal endothelial surface in a direction perpendicular to the vessel axis and parallel to adjacent smooth muscle cells. Therefore, we term the cell morphology observed here “pericyte-like” as opposed to “perivascular-like,” which encompasses both pericyte and smooth muscle cell morphology. hLFs at this time point also exhibit this morphology to a statistically similar extent (FIG. 3). This cell behavior by hASCs and hLFs was observed to a much lesser degree after 30 days, but increased after 60 days in vivo (FIG. 3), and was independent of cell passage number (P2-P4) at the time of injection (data not shown). After 60 days, a significantly higher number of hASCs than hLFs exhibited pericyte-like morphologies in tissues treated with Compound 48-80 (FIG. 3). hASCs (FIG. 4A-D) and hLFs (FIG. 4E) are easily identified in the mesenteric tissue, which is a thin tissue (approximately 100 μm thick) that permits en face visualization of entire microvascular networks and single-cell resolution of microvessel and cell morphology.

Vascular Density of Microvessels is Affected by hASCs at Early and Late Time Points

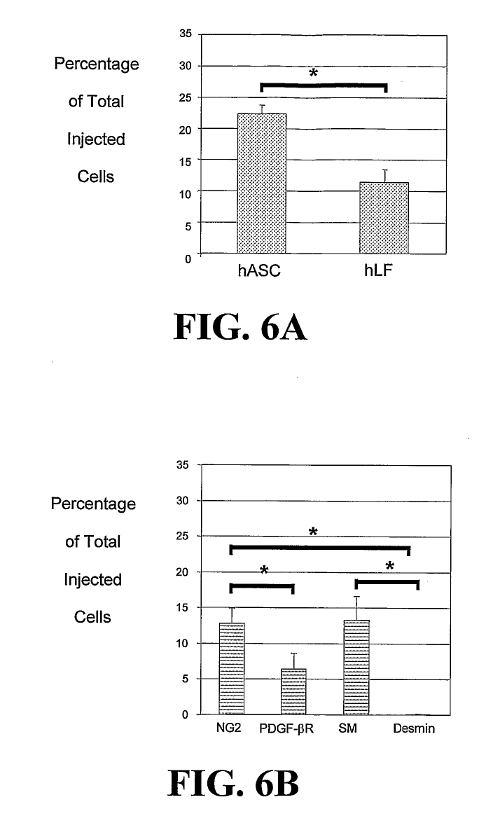

Because hASCs exhibited pericyte-like morphologies in vivo and native pericytes in the microvasculature have been shown to contribute to microvessel maintenance and prevent vascular regression, we investigated the functional effect of adipose-derived cells on microvascular length density in networks stimulated to undergo remodeling. Ten days after hASC injection, length density, or total vascular length, increased significantly in tissues treated with hASCs relative to tissues treated with hLFs, regardless of 48-80 stimulation (FIG. 5A-B). Length densities of hASC-treated tissues were also significantly elevated above tissues receiving vehicle control (no cells) in tissues not treated with Compound 48-80. Compound 48-80 stimulation, which is known to evoke peak remodeling responses between 14 and 20 days after delivery (Norrby et al., Virchows Archiv. B. Cell Pathology. 52:195, 1986), increased vascular length density relative to untreated tissues, except in tissues receiving hLFs (FIG. 5A-B), suggesting that the presence of hLFs in the tissue might delay or inhibit the stimulated vascular remodeling response.

After 30 days, there was no significant difference in length density between treatment groups in 48-80 stimulated or un-stimulated tissues (FIG. 5A-B). This is also to be expected based on the transient nature of the remodeling response to Compound 48-80, whereby after the peak remodeling response newly sprouted vessels gradually regress, causing length density to return to control levels after the inflammatory response has subsided.

By day 60, the presence of hASCs (in both Compound 48-80 stimulated and un-stimulated tissues) evokes significant length density increases compared to that of hLF-treated and vehicle control-treated tissues (FIG. 5A-B). This increase in length density coincides temporally with the elevated percentage of hASCs exhibiting a pericyte-like morphology compared to hLFs (FIG. 3), suggesting that the two are causally related. This supports the hypothesis that the presence of hASCs in the tissue at late time points provides longer lasting support to vessels that would normally regress in vehicle control- or hLF-treated tissues. Furthermore, in tissues not treated with Compound 48-80, vascular length density at day 60 is elevated to levels comparable to those measured on day 10 (FIG. 5B) and significantly larger than in tissues treated with Compound 48-80 at day 60 (FIG. 5A).

Although HASC numbers (Table 1) suggest that the inflamed tissue environment created by Compound 48-80 stimulation is supportive of cell proliferation, or cell survival/migration, this vascular density result suggests that in Compound 48-80 un-treated tissues, the cells that survive or migrate in the in vivo environment, although fewer, can perhaps elicit a more potent vascular density response at later time points. This is further supported by the fact that Compound 48-80 has direct deleterious effects on HASC viability in vitro.

hASCs Express Perivascular Cell Markers In Vivo

To determine if injected hASCs expressed markers consistent with a perivascular cell phenotype, tissues were immunostained for an array of markers known to be expressed by smooth muscle cells and pericytes: NG2, SMA, PDGF-β receptor, and desmin (Gerhardt and Betsholtz, Cell Tissue Res. 314:15, 2003; Murfee et al., Microcirculation 12:151, 2005). Although we never observed hASCs or hLFs in an endothelial cell location or via co-labeling with BSI-lectin (an endothelial cell marker), hASCs express a number of perivascular cell (pericytes and smooth muscle cell) markers in vivo according to immunohistochemistry (Table 2). A significantly larger number of hASCs express NG2 and SMA relative to hLFs after 60 days. Of the total number of hASCs in the tissue, 8% both express NG2 and exhibit pericyte-like morphologies, while 10% express SMA and exhibit pericyte-like morphologies (Table 2, FIG. 4A-D).

TABLE 2
Expression of pericyte markers by hASC and hLF populations 60
days after injection into Compound 48-80-stimulated tissue

| | | | | | Percentage of total | |
|---|---|---|---|---|---|---|
| | | | | | injected cells both | |
| | | | | | exhibiting pericyte- | |
| | Percentage of total injected | | Percentage of total injected | | like morphology and | |
| | cells expressing pericyte | | cells exhibiting pericyte- | | expressing pericyte | |
| Pericyte | markers | | like morphology | | markers | |
| Marker | hASCs | hLFs | hASCs | hLFs | hASCs | hLFs |
| NG2 | 21 ± 3* | 0 ± 0 | 32 ± 3* | 15 ± 4 | 8 ± 1* | 0 ± 0 |
| PDGF-bR | 11 ± 4 | N/A | 23 ± 3 | N/A | 5 ± 2 | N/A |
| SMA | 23 ± 4* | 9 ± 5 | 37 ± 3* | 9 ± 2 | 10 ± 3* | 0 ± 0 |
| Desmin | 0.1 ± 0.1 | 0 ± 0 | 21 ± 3 | 10 ± 2 | 0.1 ± 0.1 | 0 ± 0 |

*= significantly different from hLFs, (±Standard error, N/A = not assessed, p ≦ 0.05).

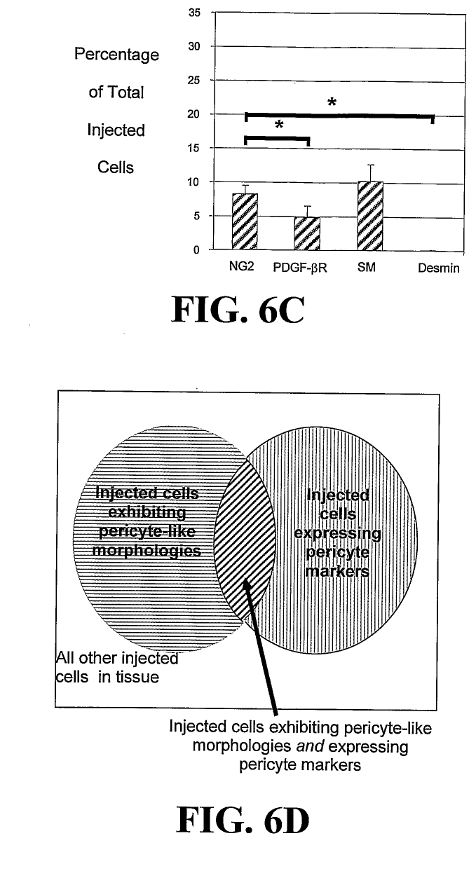

Also quantified was the relationship between perivascular marker expression and pericyte-like morphologies by injected human cells (FIG. 6D). Significantly, more hASCs than hLFs in the tissue exhibited pericyte-like morphologies (FIG. 6A). Of hASCs not exhibiting a pericyte-like morphology, significantly more expressed NG2 compared to those that expressed PDGF-β receptor and desmin. SMA was expressed by this cell population to a similar extent (FIG. 6B). A significantly greater number of hASCs exhibiting pericyte-like morphologies also expressed NG2 compared to those expressing PDGF-β receptor and desmin (FIG. 6C). NG2 is expressed by quiescent (Murfee et al., Microcirculation 12:151, 2005) and remodeling pericytes and smooth muscle cells (Gerhardt and Betsholtz, Cell Tissue Res. 314:15, 2003), and the fact that it is expressed by injected hASCs at significant levels—specifically those cells that also exhibit pericyte-like morphologies—suggests that a subfraction of hASCs may have the potential to differentiate into perivascular cells.

Correlation between hASCs Perivascular Cell Marker Expression. Morphology and Vascular Density

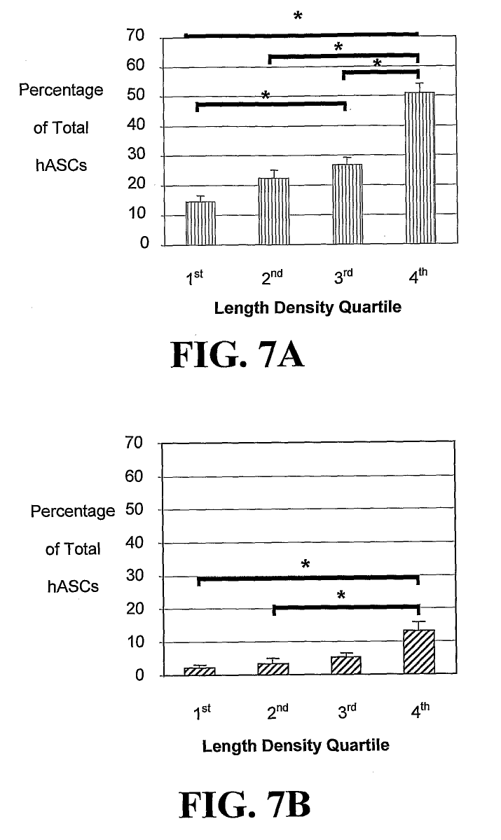

To determine if hASC pericyte-like morphology and marker expression were correlated with changes in vascular length density, the percentage of total hASCs exhibiting pericyte-like morphologies (FIG. 7A) and the percentage of total hASCs exhibiting pericyte-like morphologies and expressing perivascular cell markers (FIG. 7B) was plotted against vessel length density quartiles (for example, the 4^{th} quartile represents data from the tissue specimens whose vascular length densities fall in the top 25th percentile). These data suggest that there is a significant correlation between vascular length density and pericyte-like behavior (expression and morphology). hLFs did not exhibit these correlations with vascular length density (data not shown).

Headings are included herein for reference and to aid in locating certain sections. These headings are not intended to limit the scope of the concepts described therein under, and these concepts may have applicability in other sections throughout the entire specification.

The disclosures of each and every patent, patent application, and publication cited herein are incorporated herein by reference in their entirety.

Other methods which were used but not described herein are well known and within the competence of one of ordinary skill in the art of clinical, chemical, cellular, histochemical, biochemical, molecular biology, microbiology and recombinant DNA techniques.

The description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein, but is to be accorded the widest scope consistent with the principles and novel features disclosed herein. The appended claims are intended to be construed to include all such embodiments and equivalent variations.

What is claimed is:

1. An isolated adipose tissue-derived stromal cell, wherein said cell is a perivascular cell or can differentiate into a perivascular-like cell.

2. The cell of claim 1, wherein said perivascular-like cell is a pericyte-like cell.

3. The cell of claim 2, wherein said pericyte-like cell is a pericyte.

4. The cell of claim 2, wherein said cell expresses at least one protein selected from the group consisting of NG2, desmin, PDGF-β receptor, and SMA.

5. The cell of claim 4, wherein said cell further comprises pericyte morphology.

6. The cell of claim 1, wherein said cell is a human cell.

7. The cell of claim 1, wherein said cell can differentiate into a perivascular-like cell in vivo.

8. A method of obtaining isolated perivascular-like precursor cells from adipose tissue, said method comprising:
a. obtaining adipose tissue from a subject;
b. separating adipocytes from stromal cells;
c. preparing said stromal cells for cell culture; and
d. plating said stromal cells derived from said prepared adipose tissue into cell culture plates comprising growth medium, thereby obtaining isolated perivascular-like precursor cells.

9. The method of claim 8, wherein said adipose tissue is human adipose tissue.

10. The method of claim 9, wherein said perivascular-like precursor cell can differentiate into a perivascular-like cell.

11. The method of claim 10, wherein said perivascular-like cell is a perivascular cell.

12. The method of claim 11, wherein said perivascular cell is a pericyte-like cell.

13. The method of claim 12, wherein said pericyte-like cell is a pericyte.

14. The method of claim 13, wherein said pericyte-like cells expresses at least one pericyte marker.

15. The method of claim 14, wherein said pericyte marker is selected from the group consisting of NG2, desmin, PDGF-β receptor, and SMA.

16. The method of claim 15, wherein said cells proliferate in culture.

17. The method of claim 8, wherein said culture medium is serum-free.

18. A method of producing a perivascular-like cell, said method comprising administering a cell of claim 1 to a subject, thereby producing a perivascular-like cell.

19. The perivascular-like cell of claim 18, wherein said cell is a pericyte-like cell.

20. A method of treating a subject with human adipose tissue-derived perivascular-like cells or perivascular precursor cells, wherein said subject is suffering from a disease, disorder, condition, or injury characterized by a need for perivascular cells, said method comprising the steps of:
a. obtaining adipose tissue comprising cells capable of differentiating into perivascular-like cells; and
b. administering to said subject a composition comprising an amount of said cells to treat said disease, disorder, condition, or injury,
thereby treating said disease, disorder, condition, or injury.

21. The method of claim 20, wherein said adipose tissue is cultured prior to administration to said subject.

22. The method of claim 21, wherein said treatment enhances vascular remodeling.

23. The method of claim 22, wherein said vascular remodeling comprises increased vascular density.

24. The method of claim 21, wherein said cells are pretreated with at least one pericyte differentiation-inducing compound prior to administration to said subject.

26. The method of claim 21, wherein said composition comprises a delivery vehicle.

27. The method of claim 21, wherein said cells have been modified to alter gene expression.

28. A method of identifying a compound which modulates perivascular-like cell production, said method comprising contacting a culture comprising cells of claim 1 with a test compound and administering said cells to a first subject, comparing the level of perivascular-like cell production of said cells in said first subject to the level of perivascular-like cell production of otherwise identical cells not contacted with said test compound administered to a second subject, wherein a higher or lower level of perivascular-like cell production in said first subject, compared with the level of perivascular-like cell productions in said second subject, is an indication that said test compound modulates perivascular-like cell production, thereby identifying a compound which modulates perivascular-like cells production.

29. The method of claim 29, wherein said perivascular-like cell is a pericyte-like cell.

30. The method of claim 29, wherein said compound stimulates perivascular-like cell production.

31. The method of claim 29, wherein said compound inhibits perivascular-like cell production.

32. A compound identified by the method of claim 28.

33. A method of treating a subject suffering from a disease, disorder, condition, or injury characterized by a need to regulate microvasculature growth and remodeling, said method comprising administering to said subject a therapeutically-effective amount of a compound identified by the method of claim 28.

34. The method of claim 33, wherein said compound is co-administered with perivascular-like precursor cells.

35. A method of producing a tissue-engineered vascular construct, said method comprising co-culturing adipose tissue-derived stromal cells with endothelial cells, wherein said stromal cells are perivascular cells or perivascular precursor cells, and optionally co-culturing said stromal cells and endothelial cells with at least one other cell type, thereby producing a tissue-engineered vascular construct.

36. The method of claim 35, wherein said other cell types are selected from the group consisting of smooth muscle cells and fibroblasts.

37. The method of claim 35, wherein said tissue-engineered vascular construct is produced in combination with a tissue-engineered construct to provide blood supply to said tissue-engineered construct, said method comprising combining the components of the tissue-engineered vascular construct, or the tissue-engineered vascular construct produced therefrom, with the cells used to produce the tissue-engineered construct, or with the tissue-engineered construct produced therefrom.
